(19) United States
(12) Patent Application Publication
(10) Pub. No.: US 2013/0096546 A1
(43) Pub. Date: Apr. 18, 2013

(54) NON-UNIFORM BEAM OPTICAL TREATMENT METHODS AND SYSTEMS

(71) Applicants: Mirko Georgiev Mirkov, Chelmsford, MA (US); Rafael Armando Sierra, Palmer, MA (US); George E.S. Cho, Hopkinton, MA (US)

(72) Inventors: Mirko Georgiev Mirkov, Chelmsford, MA (US); Rafael Armando Sierra, Palmer, MA (US); George E.S. Cho, Hopkinton, MA (US)

(73) Assignee: Cynosure, Inc., Westford, MA (US)

(21) Appl. No.: 13/508,293

(22) PCT Filed: Mar. 5, 2010
(86) PCT No.: PCT/US10/26432

§ 371 Date: Aug. 14, 2012

Related U.S. Application Data
(60) Provisional application No. 61/157,862, filed on Mar. 5, 2009.

(51) Int. Cl. A61B 18/20

(52) U.S. Cl. CPC A61B 18/203
USPC 606/9

(57) ABSTRACT

An apparatus is disclosed including: an incoherent light source that generates a treatment beam having a non-uniform energy profile, the non-uniform energy profile being included of regions of relatively high energy per unit area within a substantially uniform background region of relatively low energy per unit area.

### RELATED APPLICATION

The present application claims benefit of U.S. Provisional Patent Application Ser. No. 61/157,862 filed Mar. 5, 2009, the entire contents of which is incorporated by reference herein in its entirety.

This application is a continuation in part of U.S. patent application Ser. No. 11/347,672 filed Feb. 3, 2006 which in turn claims the benefit of U.S. Provisional Application No. 60/673,914, filed Apr. 22, 2005, the entire contents of each of which are incorporated herein by reference.

### BACKGROUND OF THE INVENTION

Plastic surgeons, dermatologists and their patients continually search for new and improved methods for treating the effects of an aging skin. One common procedure for rejuvenating the appearance of aged or photodamaged skin is laser skin resurfacing using a carbon dioxide laser. The carbon dioxide laser energy is absorbed by tissue water causing vaporization of the outer skin layer. Carbon dioxide lasers have been utilized for approximately three decades. However it has only been the past few years that these lasers have been arranged to remove only thin tissue layers with minimal heat damage to the surrounding skin. While carbon dioxide lasers may remove about 150 microns of skin, that skin may take a month or more to heal under such a procedure.

Er:YAG lasers have been utilized to ablate even thinner layers of tissue than carbon dioxide layers. However they lack the coagulation characteristics and thus allow more bleeding than a carbon dioxide laser during use.

Non-ablative skin rejuvenation is a methodology which does not take the top layer of skin off, but which uses a deep-penetrating laser to treat the layers of skin beneath the outer epidermal layer, treating unsightly vascular and pigmented lesions, and shrinking and modifying the underlying collagen, tightening the skin and reducing wrinkles to provide a more youthful appearance. This methodology however, has a low efficiency, and an aggressive cooling method must be used on to the skin so as to minimize damaging the top or upper layer thereof and also to minimize pain generation. The “fluence” or energy density used is greater than 10 joules per square centimeter and to be more effective this fluence often reaches 30 Joules per square centimeter. This level of energy often causes pain and epidermal damage.

United States Published Patent Application No. 2002/0161357 A1, by Anderson et al., discusses a method and apparatus for performing therapeutic treatment on a patient's skin by using focused radiation beams to create “islands” of treatment/damage within untreated portions of the patient's skin. However, the parameters of the treatment beam in this method are not optimal for skin rejuvenation treatment.

Yet another treatment method is disclosed in U.S. Pat. No. 6,077,294 to Cho et al., the entire teachings of which are incorporated herein by reference. This patent describes a system and methodology for noninvasive skin treatment that utilizes a pulsed dye laser having a wavelength of about 585 nanometers (nm), and an energy of less than 5 Joules per square cm. In contrast to earlier techniques which used higher-energy pulses to damage and “shrink” the collagen below the epidermis, the relatively lower energies of the beams in the '294 patent are designed to stimulate the collagen to regenerate and “fill in” valleys of the skin for a younger more clearer skin.

### SUMMARY OF THE INVENTION)

The present invention relates to methods and apparatus for treatment using non-uniform laser radiation. Preferably, the invention is used for skin rejuvenation treatment, in which a high-intensity portion of the laser radiation causes collagen destruction and shrinkage within select portions of the treatment area, while a lower-intensity portion of the radiation causes fibroblast stimulation leading to collagen production across other portions of the treatment area.

In some embodiments, the method and system of the invention utilize a solid-state laser source, such as an Nd:YAG laser. The output beam from the laser source is coupled into an optical system that modifies the beam to provide a large-diameter beam having a non-uniform energy profile, comprised of a plurality of high-intensity zones surrounded by lower-intensity zones within the treatment beam. The higher-intensity zones heat select portions of the target tissue to temperatures sufficient for a first treatment (e.g. collagen shrinkage), while the lower-intensity zones provide sufficient energy to the surrounding tissue for a second treatment (e.g. stimulated collagen production). Thus, a large area of tissue, preferably 7-10 mm in diameter, can be treated simultaneously, while minimizing the risk of burning or other damage to the skin.

In one embodiment, the invention uses a fiber bundle to provide a non-uniform energy output beam. In another embodiment, the invention uses a diffractive lens array to produce the non-uniform output beam.

A method of treating human skin in accordance with one aspect of the invention comprises generating an output beam from a laser source, such as an Nd:YAG laser; coupling the beam into an optical system that modifies the beam to provide a treatment beam having a non-uniform energy profile, the treatment beam comprised of a plurality of high-intensity zones surrounded by low-intensity zones within the treatment beam; and directing the treatment beam to a target tissue area such that the high-intensity zones heat select portions of the target tissue to temperatures sufficient for a first treatment, while the lower-intensity zones provide sufficient energy to the surrounding tissue for a second treatment. Preferably, the first treatment comprises collagen shrinkage and the second treatment comprises collagen stimulation. The output beam can have a wavelength between about 1.3 to 1.6 microns, and preferably between about 1.41 and 1.44 microns, and a pulse duration between 0.1 and 100 milliseconds, and preferably between about 1 and 5 milliseconds. The average fluence of the treatment beam can be less than about 10 J/cm². Generally, the average fluence of the treatment beam is between about 5-6 J/cm². The average fluence in the lower-intensity zones is generally on the order of 2-3 J/cm².

The optical system can comprise a fiber bundle, having 1000 to 2000 separate fibers, for instance, and a focusing lens for coupling the beam into the fiber bundle. An optical window, preferably between 1 and 5 mm thick, can be located at the distal end of the bundle, the optical window permitting the beams emitted from each fiber in the bundle to diverge and partially overlap with one another before they reach the target tissue. In certain embodiments, a transport fiber can carry the output beam from the laser source to the fiber bundle, and the fiber bundle can be located in a handpiece.

In another embodiment, the optical system can comprise a diffractive lens array, preferably comprised of about 2000 or less lenses, arranged in an optical path between a laser source and the treatment area, such that each lens in the array provides a high-intensity zone surrounded by a low intensity zone of radiation. Each lens in the array can have a diameter of between about 150 and 450 microns, and the entire lens array can have a diameter of between about 7 and 10 mm. Preferably, the average fluence of the laser output beam is less than about 10 J/cm².

In another embodiment, a laser system of the invention comprises a laser source that generates an output beam; and an optical system that modifies the output beam to provide a treatment beam having a non-uniform energy profile, the treatment beam being comprised of a plurality of high-intensity zones surrounded by low-intensity zones within the treatment beam, such that the high-intensity zones heat select portions of a target tissue to temperatures sufficient for a first treatment, while the lower-intensity zones provide sufficient energy to the surrounding tissue for a second treatment. The laser source can be an Nd:YAG laser, and generally produces an output beam having a wavelength between about 1.3 to 1.6 microns, and preferably between about 1.41 and 1.44 microns, and a pulse duration between 0.1 and 100 milliseconds, preferably between about 1 and 5 milliseconds. The optical system can comprise a fiber bundle, preferably with an optical window between the distal end of the bundle and the target tissue. Alternatively, the optical system can include a diffractive lens array in the optical path between the source and the treatment area, such that each lens in the array provides a high-intensity zone surrounded by a low intensity zone of radiation.

According to another embodiment, a laser system comprises a laser source that generates an output beam; a fiber bundle comprising a plurality of individual fibers, the fiber bundle having a proximal end and a distal end; a focusing lens for coupling the output beam into a proximal end of the fiber bundle; and an optical window at the distal end of the fiber bundle, the optical window permitting the beams emitted from each fiber in the bundle to diverge as the beam passes through the optical window so that each beam partially overlaps with the beam(s) from adjacent fibers in the bundle. The optical window can comprise a transparent material, such as glass, or could comprise a spacer having an empty space between the distal end of the fiber bundle and the treatment area.

According to yet another embodiment, a laser system comprises a laser source that generates an output beam; and a diffractive lens array arranged in an optical path between a laser source and a treatment area, such that each lens in the array provides a high-intensity zone surrounded by a low intensity zone of radiation.

In another aspect, an apparatus is disclosed including: an incoherent light source that generates a treatment beam having a non-uniform energy profile, the non-uniform energy profile being included of regions of relatively high energy per unit area within a substantially uniform background region of relatively low energy per unit area.

In some embodiments, the treatment beam is configured such that the regions of relatively high energy per unit area deliver sufficient energy to target tissue to heat select portions of the target tissue to a first temperature to shrink collagen. The substantially uniform background region of relatively low energy per unit area delivers sufficient energy to target tissue to stimulate collagen production in the remaining portion of the target tissue

Some embodiments include an optical system that receives at least a portion of an output light beam from the incoherent light source, and modifies the portion of the output beam to provide the treatment beam having a non-uniform energy profile.

In some embodiments, the optical system includes a fiber bundle including multiple optical fibers, where each of the optical fibers in the fiber bundle has an input face adapted to receive only a portion of the output beam.

In some embodiments, the fiber bundle includes 1000 to 2000 fibers.

In some embodiments, the optical system includes a focusing lens for coupling the output beam into a proximal end of the fiber bundle, and an optical window between the distal end of the fiber bundle and a target plane, the optical window permitting the beam emitted from each fiber in the bundle to diverge before it reaches the target plane that each beam partially overlaps with one or more beams from adjacent fibers in the bundle.

In some embodiments, the optical system includes a diffractive lens array including multiple diffractive lenses arranged in an optical path between the at least partially coherent source and a treatment plane, such that each lens in the array provides a high-intensity zone surrounded by a low intensity zone of radiation at the treatment plane, and where each of the diffractive lenses is adapted to receive only a portion of the output beam.

In some embodiments, the diffractive lens array has more than about 1000 and less than about 2000 diffractive lenses.

In some embodiments, the incoherent source includes an LED.

In some embodiments, the wavelength of the treatment beam is between about 1.3 microns and 1.6 microns or between 1.40 and 1.44 microns.

In some embodiments, e the incoherent source includes an LED array. In some embodiments, the LED array includes a plurality of spatially separated LED emitters positioned to produce the plurality of high-intensity zones surrounded by low-intensity zones within the treatment beam. In some embodiments, the plurality of spatially separated LEDs are arranged in a grid. In some embodiments, the LED array is mounted on a heat dissipating substrate. In some embodiments, the LED array includes: a first plurality of LEDs adapted to emit light at a first intensity; and a second plurality LEDs adapted to emit light at a second intensity less than the first intensity, where the first and second plurality of LEDs are interspersed to produce the treatment beam included of a plurality of high-intensity zones surrounded by low-intensity zones within the treatment beam.

In some embodiments, the LED array is a densely packed array. In some embodiments, the LED) array includes multiple led emitters, and the plurality of high-intensity zones surrounded by low-intensity zones within the treatment beam are produced by varying one or more of the following across the LED array: LED emitter size, LED emitter output intensity, LED emitter spacing.

In some embodiments, the ratio of peak energy per unit area in the regions of relatively high energy per unit area to the average energy per unit area in the background region is greater than 4.5 to 1, greater than 10 to 1, greater than 50 to 1, greater than 100 to 1, or greater than 150 to 1.

In another aspect, a method of treating human tissue is disclosed, including: generating a treatment beam having a non-uniform energy profile from an incoherent light source, the non-uniform energy profile being included of regions of relatively high energy per unit area within a substantially uniform background region of relatively low energy per unit area; and directing the treatment beam to a target tissue area such that such that the regions of relatively high energy per unit area deliver sufficient energy to target tissue to heat select portions of the target tissue to a first temperature to shrink collagen and where the substantially uniform background region of relatively low energy per unit area delivers sufficient energy to target tissue to stimulate collagen production in the remaining portion of the target tissue.

In some embodiments, the least partially incoherent light source includes an LED array.

In some embodiments, the wavelength of the treatment beam is between about 1.3 microns and 1.6 microns or between 1.40 and 1.44 microns.

In some embodiments, the ratio of peak energy per unit area in the regions of relatively high energy per unit area to the average energy per unit area in the background region is greater than 4.5 to 1, greater than 10 to 1, greater than 50 to 1, greater than 100 to 1, or greater than 150 to 1.

In another aspect, a method of treating human tissue is disclosed including: providing one or more initial treatments to a target tissue area, each laser treatment including: generating an output beam from a laser source; coupling the beam into an optical system that modifies the beam to provide a laser initial treatment beam having a non-uniform energy profile, the initial treatment beam non-uniform energy profile being included of regions of relatively high energy per unit area within a substantially uniform background region of relatively low energy per unit area; and directing the initial treatment beam to a target tissue area such that the regions of relatively high energy per unit area deliver sufficient energy to target tissue to heat select portions of the target tissue to a first temperature to shrink collagen and where the substantially uniform background region of relatively low energy per unit area delivers sufficient energy to target tissue to stimulate collagen production in the remaining portion of the target tissue. The method further includes after providing the one or more initial treatments, providing one or more maintenance treatments, each maintenance treatment including: generating a maintenance treatment beam having a non-uniform energy profile from an incoherent light source, the maintenance treatment beam non-uniform energy profile being included of regions of relatively high energy per unit area within a substantially uniform background region of relatively low energy per unit area; and directing the maintenance treatment beam to a target tissue area such that the regions of relatively high energy per unit area deliver sufficient energy to target tissue to heat select portions of the target tissue to a first temperature to shrink collagen and where the substantially uniform background region of relatively low energy per unit area delivers sufficient energy to target tissue to stimulate collagen production in the remaining portion of the target tissue.

In some embodiments, the incoherent light source includes an LED array. In some embodiments, the wavelength of the initial treatment beam or the maintenance treatment beam is between about 1.3 microns and 1.6 microns or between 1.40 and 1.44 microns.

In another aspect, a method of treating human tissue including an first layer of tissue overlaying a second layer of tissue is disclosed, the method including: generating a treatment beam having a non-uniform energy profile from a light source, the non-uniform energy profile being included of regions of relatively high energy per unit area within background region of relatively low energy per unit area; directing the treatment beam to impinge on the first layer of tissue to form one or more sacrificial channels of damaged tissue in the first layer at positions corresponding to regions of relatively high energy per unit area, where the sacrificial channels are surrounded by regions of substantially undamaged tissue in the first layer at positions corresponding to regions of relatively high energy per unit area; and transmitting treatment beam light through the sacrificial channels to the second layer.

Some embodiments include scattering at least a portion of the treatment beam light transmitted to the second layer to direct the portion of light to locations in the second layer underlying the regions of undamaged tissue in the first layer. In some embodiments, the second layer of tissue includes one or more target structures, and further including directing at least a portion of the treatment beam light transmitted to the second layer to the target structures. In some embodiments, the target structure includes at least one from the list consisting of: a foreign body, a tattoo ink particle, a sebaceous gland, a hair follicle, a blood vessel, and region of lipid rich tissue.

In some embodiments, the first layer includes an epidermis of a region of skin and the second layers include a dermis of a layer of skin.

In some embodiments, the ratio of peak energy per unit area in the regions of relatively high energy per unit area to the average energy per unit area in the background region is greater than 4.5 to 1, greater than 10 to 1, greater than 50 to 1, greater than 100 to 1, or greater than 150 to 1.

In some embodiments, the regions of relatively high energy per unit area include about 20% or less of the total area of a cross section of the treatment beam at the first layer.

Some embodiments include stimulating collagen generation the regions of substantially undamaged tissue in the first layer in response to light from the regions of relatively low energy per unit area of the treatment beam.

In another aspect, a method of treating human tissue including an first layer of tissue overlaying a second layer of tissue is disclosed, the method including: generating a first light beam at a first wavelength, the first light beam having a non-uniform energy profile, the non-uniform energy profile being included of regions of relatively high energy per unit area within background region of relatively low energy per unit area; directing the first treatment beam to impinge on the first layer of tissue to ablate tissue in the first layer at positions corresponding to regions of relatively high energy per unit area to form channels extending at least partially through the first layer; generating a second light beam at a second wavelength; and directing the second light beam to impinge on the first layer such that a portion of the second light beam is transmitted the channels to the second layer.

In some embodiments, light at the first wavelength is more preferentially absorbed by the first) layer of tissue than light at the second wavelength.

In some embodiments, the second light beam has a non-uniform energy profile, the non-uniform energy profile being includes regions of relatively high energy per unit area within background region of relatively low energy per unit area. The step of directing the second light beam to impinge on the first layer includes directing the second light beam to the first layer such that the regions of relatively high energy per unit area of the second light beam impinge upon the first layer at locations which substantially correspond to the channels in the first layer.

Some embodiments include controlling the ablation of the tissue in the first layer such that the channels extend substantially through the first layer to a location proximal an interface between the first and second layer. In some embodiments, the controlling the ablation includes controlling at least one of: an intensity of the first light beam, a pulse period of the first light beam, a pulse rate of the first light beam, a pulse shape of the first light beam.

In some embodiments, the channels are surrounded by regions of substantially undamaged tissue.

Some embodiments include scattering at least a portion of light from the second beam transmitted to the second layer to direct the portion of light to locations in the second layer which do not underlay the channels.

In some embodiments, the second layer of tissue includes one or more target structures, and further including directing at least a portion of the light from the second beam to the target structures.

In some embodiments, the target structure includes at least one from the list consisting of: a foreign body, a tattoo ink particle, a sebaceous gland, a hair follicle, a blood vessel, and region of lipid rich tissue.

In some embodiments, the first layer includes an epidermis of a region of skin and the second layer includes a dermis of a layer of skin.

In some embodiments, for the first beam, the ratio of peak energy per unit area in the regions of relatively high energy per unit area to the average energy per unit area in the background region is greater than 4.5 to 1, 10 to 1, 50 to 1, 100 to 1, or more.

In some embodiments, the regions of relatively high energy per unit area include about 20% or less of the total area of a cross section of the treatment beam at the first layer.

In another aspect, and apparatus is disclosed including: an optical delivery head; a scanner device for selectively positioning the optical delivery head at each of a plurality of locations above a treatment region including a first layer of tissue overlaying a second layer of tissue; and a controller. The controller is configured to: direct the optical delivery head to each of the locations, and at each location respectively, direct the optical delivery head to: emit a first beam of light at a first wavelength to ablate a respective channel at least partially through the first layer of tissue, and emit a second beam of light at a second wavelength to transmit a portion of the light at the second wavelength through the channel to the second layer of tissue.

In some embodiments, the first wavelength is more preferentially absorbed by the first layer of tissue than light at the second wavelength.

In some embodiments, the controller adjusts the emission of the first light beam such the ablation of the tissue in the first layer such that the respective channels extends substantially through the first layer to a location proximal an interface between the first and second layer.

In some embodiments, the controller adjusts the emission of the first light beam such the ablation of the tissue in the first layer such that the respective channels extends through the first layer and to a desired depth in the second layer.

In some embodiments, the controller adjusts at least one property of the first light beam chosen from the list consisting of an intensity of the first light beam, a pulse period of the first light beam, a pulse rate of the first light beam, a pulse shape of the first light beam, and a wavelength of the first light beam.

In some embodiments, the optical delivery head includes one or more optical elements which limits the spot size of the first beam such that the respective channels are surrounded by regions of substantially undamaged tissue.

In another aspect, a method of treating human tissue including an first layer of tissue overlaying a second layer of tissue is disclosed, the method including: positioning the optical delivery head) at each of a plurality of locations above the first layer of tissue; and at each location respectively, emitting a first beam of light at a first wavelength from the delivery head to ablate a respective channel at least partially through the first layer of tissue; and emitting a second beam of light at a second wavelength from the delivery head to transmit a portion of the light at the second wavelength through the channel to the second layer of tissue.

In some embodiments, light at the first wavelength is more preferentially absorbed by the first layer of tissue than light at the second wavelength.

Some embodiments include controlling the emission of the first light beam such the ablation of the tissue in the first layer such that the respective channels extends substantially through the first layer to a location proximal an interface between the first and second layer.

Some embodiments include controlling the emission of the first light beam such the ablation of the tissue in the first layer such that the respective channels extends through the first layer and to a desired depth in the second layer.

In some embodiments, the controlling the emission of the first light beam includes adjust at least one property of the first light beam chosen from the list consisting of an intensity of the first light beam, a pulse period of the first light beam, a pulse rate of the first light beam, a pulse shape of the first light beam, and a wavelength of the first light beam.

Some embodiments include limiting the spot size of the first beam such that the respective channels are surrounded by regions of substantially undamaged tissue.

Some embodiments include forming an array of the channels over an array of tissue, where the area of the channels is less than about 20% of the area of tissue.

In another aspect, an apparatus is disclosed including: an optical scanner device configured to selectively direct light to each of plurality of locations of a treatment region including a first layer of tissue overlaying a second layer of tissue; a controller configured to, for each of the plurality of locations: direct a first beam of light at a first wavelength from the scanner to the first layer of tissue to ablate a respective channel at least partially through the first layer of tissue; and direct a second beam of light at a second wavelength from the scanner to the first layer of tissue and through the channel to the second layer of tissue.

In some embodiments, the light at the first wavelength is more preferentially absorbed by the first layer of tissue than light at the second wavelength.

In some embodiments, the controller adjusts the first light beam such that the respective channel extends substantially through the first layer to a location proximal an interface between the first and second layer.

In some embodiments, the controller adjusts the first light beam such that the respective channel extends through the first layer and to a desired depth in the second layer.

In some embodiments, the controller is adapted to adjust at least one property of the first light beam chosen from the list consisting of an intensity of the first light beam, a pulse period of the first light beam, a pulse rate of the first light beam, a pulse shape of the first light beam, and a wavelength of the first light beam.

In some embodiments, the scanner includes one or more optical elements which limits the spot size of the first beam such that the respective channels are surrounded by regions of substantially undamaged tissue.

In another aspect, a method of treating human tissue including an first layer of tissue overlaying a second layer of tissue is disclosed, the method including: using an optical scanner to direct light at each of a plurality of locations above the first layer of tissue; and at each location respectively, directing a first beam of light at a first wavelength to the location to ablate a respective channel at least partially through the first layer of tissue; and directing a second beam of light at a second wavelength to the location to transmit a portion of the light at the second wavelength through the channel to the second layer of tissue.

In some embodiments, light at the first wavelength is more preferentially absorbed by the first layer of tissue than light at the second wavelength.

Some embodiments include controlling the first light beam such the ablation of the tissue in the first layer such that the respective channels extends substantially through the first layer to a location proximal an interface between the first and second layer.

Some embodiments include controlling the first light beam such the ablation of the tissue in the first layer such that the respective channels extend through the first layer and to a desired depth in the second layer. In some embodiments, controlling the first light beam includes adjusting at least one property of the first light beam chosen from the list consisting of an intensity of the first light beam, a pulse period of the first light beam, a pulse rate of the first light beam, a pulse shape of the first light beam, and a wavelength of the first light beam.

Some embodiments include limiting the spot size of the first beam such that the respective channels are surrounded by regions of substantially undamaged tissue.

Some embodiments include forming an array of the channels over an array of tissue, where the area of the channels is less than about 20% of the area of tissue.

Various embodiments may include any of the above described features, either alone or in any suitable combination.

It is to be understood that the phrase “incoherent light source” refers to any non-laser light source including, but not limited to, sources made up of one or more or a combination of light emitting diodes (LED), pulsed lamps, micro-ring resonators or other emitters of electromagnetic radiation. It is also to be understood that as used herein, the terms “light” and “optical” refer not only to electromagnetic radiation in the visible spectrum, but to electromagnetic radiation in any frequency range including, ultraviolet and infrared.

The present invention provides a laser treatment which covers a large area of the patient, is characterized by high-absorption of the laser radiation and lower peak energies, which results in minimal risk of skin damage. In one aspect, the present invention advantageously accomplishes stimulated collagen production as well as collagen shrinkage simultaneously in a single treatment area. In addition to skin rejuvenation treatment, the principles of the invention can also be extended for use in other types of optical radiation treatments, including, without limitation, treatment of acne, hair removal, and treatment of vascular or pigmented lesions.

### BRIEF DESCRIPTION OF THE DRAWINGS

FIG. 1A illustrates a laser treatment system comprising a fiber bundle and optical window;

FIG. 1B is a plot of the beam profile on the skin for the laser treatment system of FIG. 1A;

FIG. 2 illustrates a laser treatment system comprising a short fiber bundle with expanded distal face;

FIG. 3 shows a diffractive lens having four levels;

FIG. 4 shows a diffractive lens having two levels;

FIG. 5 shows a diffractive lens with eight levels;

FIG. 6 shows a diffractive lens array having a hexagonal pattern;

FIG. 7 shows a diffractive lens array having an elongated hexagonal pattern;

FIG. 8 shows a treatment beam profile for a diffractive lens array;

FIG. 9 shows a plot of the relative hot area fluence factor, F_{I}/Fₐᵥ, as a function of the relative diameter of the central hot area, d/D for a diffractive lens array in accordance with one aspect of the invention;

FIG. 10 shows the temperature profile of skin treated with a non-uniform output beam from a diffractive lens array;

FIG. 11 shows a tip of a laser treatment handpiece having a cooling mechanism.

FIG. 12 shows an LED array for providing a non-uniform beam of treatment light along with a plot of an exemplary intensity distribution at a treatment plane;

FIG. 12a shows a top down view of the LED array shown in FIG. 12;

FIG. 13 shows a dense packed LED array for providing a non-uniform beam of treatment light along with a plot of an exemplary intensity distribution at a treatment plane; an

FIG. 13a shows a top down view of the LED array shown in FIG. 13;

FIGS. 14A-C show exemplary treatment schemes using a non-uniform beam;

FIG. 15 is an energy density plot for tissue treated using a non-uniform beam; and

FIG. 16 shows an exemplary treatment scheme featuring an optical scanner.

### DETAILED DESCRIPTION OF THE INVENTION

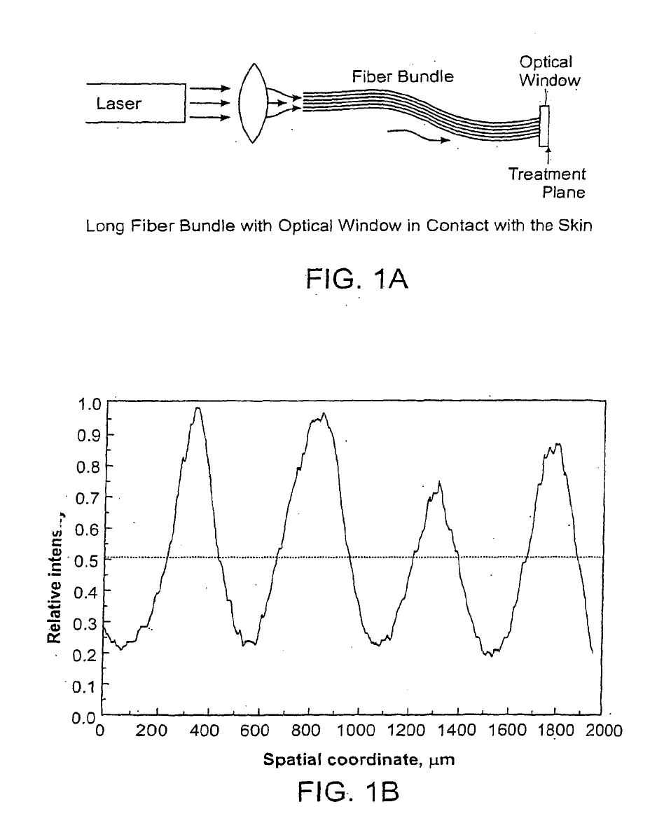

As shown in FIG. 1A, the apparatus includes a laser source that emits an output beam. The beam is coupled into a bundle of optical fibers using one or more focusing lenses. The bundle preferably contains between 1000 and 2000 separate fibers. Typically, each fiber has a diameter of about 100-200 microns. The output laser beam is thus directed to 1000-2000 smaller beams, each of which traverses the length of the fiber bundle in individual optical fibers. The fiber bundle terminates at its distal end at an optical window that can be held in direct contact with the patient's skin. The window is approximately 1-5 mm thick, and protects the output face of the fiber bundle from contamination, and also permits the beam emitted from each fiber to diverge before it reaches the patient's skin, preferably so that each beam partially overlaps with the beam(s) from adjacent fibers in the bundle.

The fibers in the bundle can be packed together tightly, or can be spaced apart from each other using mechanical spacers. The use of mechanical spacers at the distal end of the bundle spreads the energy from the bundle over a larger area, and helps to reduce the pain sensation for the patient. In general, the combined spot size on the skin from all the fibers in the fiber bundle is between approximately 7 and 10 mm in diameter.

In a preferred treatment method for the embodiment of FIG. 1A, the laser source, which is preferably an Nd:YAG laser, produces an output laser pulse having a wavelength of between 1.3 and 1.6, preferably between about 1.40 and 1.44 microns, and a pulse duration of between 0.1 and 100 milliseconds, preferably between about 1 and 5 milliseconds. Because the laser operates at wavelengths that are well-absorbed by the skin, the laser can operate at relatively low energies, and minimize the risk of burning or damage to the skin.

In operation, the optical window is held against the skin of the patient, and the laser source is energized to produce a pulse of laser light that travels from the source through the fiber bundle and the optical window, and penetrates into the patient's skin. Since the optical window is approximately 1-5 mm thick, the window also serves as a spacer between the output end of the fiber bundle and the skin of the patient. Thus, as the laser light is emitted from each fiber in the bundle, the light is permitted to diverge as it travels through the window to the patient's skin. In a preferred embodiment, the fibers are approximately 100-200 microns in diameter, and the beam emitted from each fiber, after passing through the window, produces a spot between 150-900 microns in diameter on the patient's skin. Because of the diverging nature of light emitted from an optical fiber, the light at the center of each spot will be relatively high-energy light, while the light at the periphery of each spot will have significantly lower energy. Thus, over a combined spot size of 7 to 10 mm for the entire fiber bundle, there are approximately 1000 to 2000 smaller treatment spots, generally about 150-900 microns in diameter, each consisting of a higher-fluence “hot spot” at the center of the spot surrounded by a lower-fluence “cooler zone” of radiation. The energy at the central “hot spot” is sufficient to shrink the underlying tissue, damage the collagen and produce collagen shrinkage. In general, the energy at the high-intensity zones, or “hot spots,” is sufficient to raise the temperature of the target tissue to 70° C. or higher. However, the radiation in “cooler zone” surrounding the hot spot is generally not sufficient to damage the tissue and cause collagen shrinkage in the tissue underlying these areas. In these lower-intensity “cooler zones,” the energy provided will only raise the temperature of the skin by a few degrees (or perhaps result in no appreciable temperature rise), and thus will not damage or even “shock” the tissue. However, this lower-intensity radiation is generally more appropriate or preferred to stimulate the fibroblasts in the tissue to produce collagen and “fill in” the skin for a younger more clearer skin In a preferred embodiment, the fibers in the bundle are arranged so that the spot sizes of radiation from each fiber abut or partially overlap with the spots from the adjacent fibers in the bundle on the patient's skin. In this way, the invention can simultaneously provide two modes of skin rejuvenation treatment: higher-energy collagen shrinkage treatment in the “hot spots” at the center of each output spot from the fiber bundle, and overall stimulated collagen production throughout the entire area of the combined fiber bundle output beam.

An example of a laser treatment method using a fiber bundle delivery system is illustrated in FIG. 1B, which is a plot of the relative intensity on the skin as a function of location on the skin for four fibers in the bundle. In practice, the fiber bundle will consist of 1000-2000 individual fibers, in a regularly-spaced arrangement to form a bundle. In this embodiment, the center-to-center distance between adjacent fibers in the bundle is approximately 500 microns. The diameter of each fiber is approximately 200 microns, and the numerical aperture (NA) of the fibers is approximately 0.2. The total diameter of the fiber bundle is approximately 9 millimeters.

The laser energy emitted from each fiber diverges as it passes through the transparent window, so that the spot size on the skin from each fiber is at least about 250 microns in diameter. Thus, the spots from each fiber generally abut or partially overlap with the spots from the adjacent fibers in the bundle. This is shown in FIG. 1B, where it can be seen that the whole area is treated with at least a low-intensity pulse, while the areas at the center of each spot receive a significantly higher dose of energy. The dotted line represents the average intensity throughout the treatment area. In this example, the peak fluence on the skin at the center of each spot is approximately 9 J/cm², while the fluence at the periphery of each spot is approximately 2 J/cm². The total area fluence is approximately 5 J/cm².

The fluence(s) received at various portions of the treatment area can be varied and controlled by, for instance, raising or lowering the total energy output from the laser source, changing the center-to-center distances between fibers in the bundle, using different diameter fibers, using fibers with a different NA to change the divergence of the beam and/or altering the thickness of the optical window to allow for a greater or lesser amount of beam divergence. The beam profile can thus be optimized for a variety of different conditions and laser treatment methods.

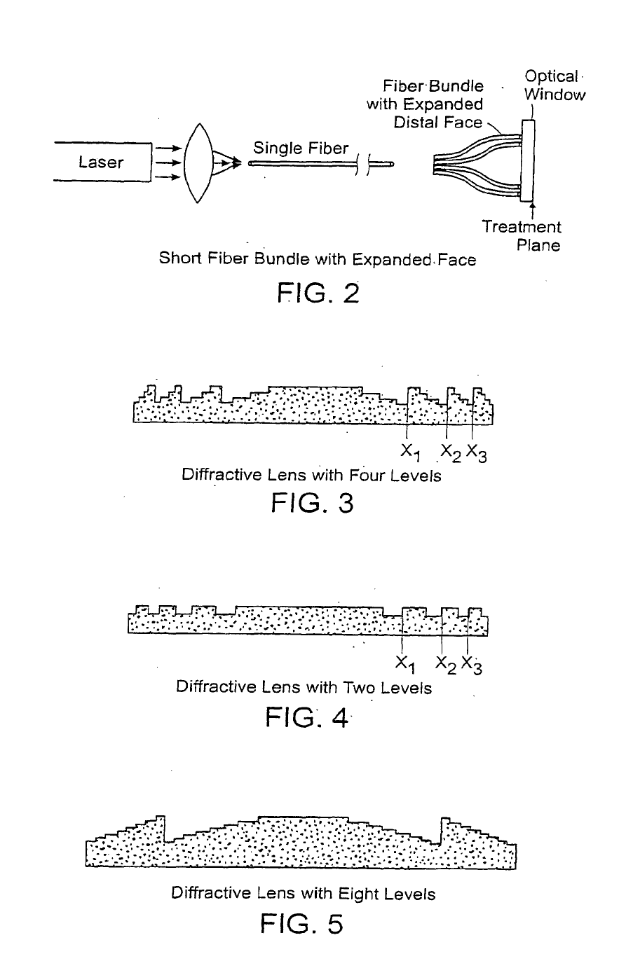

FIG. 2 shows yet another embodiment that is similar to the embodiment of FIG. 1, except that instead of a long-fiber bundle coupling the laser output beam from the source to the optical window, this embodiment uses a single transport fiber to carry the laser energy from the laser source to a handpiece containing a shorter fiber bundle. At the handpiece, the output laser pulse) from the single fiber is coupled into the short fiber bundle. As in the prior embodiment, the short fiber bundle is comprised of a plurality of separate optical fibers, preferably 1000 to 2000 fibers. The short fiber bundle has a smaller bundle diameter at its proximal end to allow the output light from the single transport fiber to efficiently couple into the bundle. The fiber bundle “fans out” from its proximal end to its distal end, using, for example, mechanical spacers, to provide an expanded face at it's output. Preferably, the expanded face has a diameter of between approximately 7 to 10 mm, and is coupled to an optical window, as in the embodiment of FIG. 1. The embodiment of FIG. 2 preferably uses the same treatment parameters as those described in connection with FIG. 1.

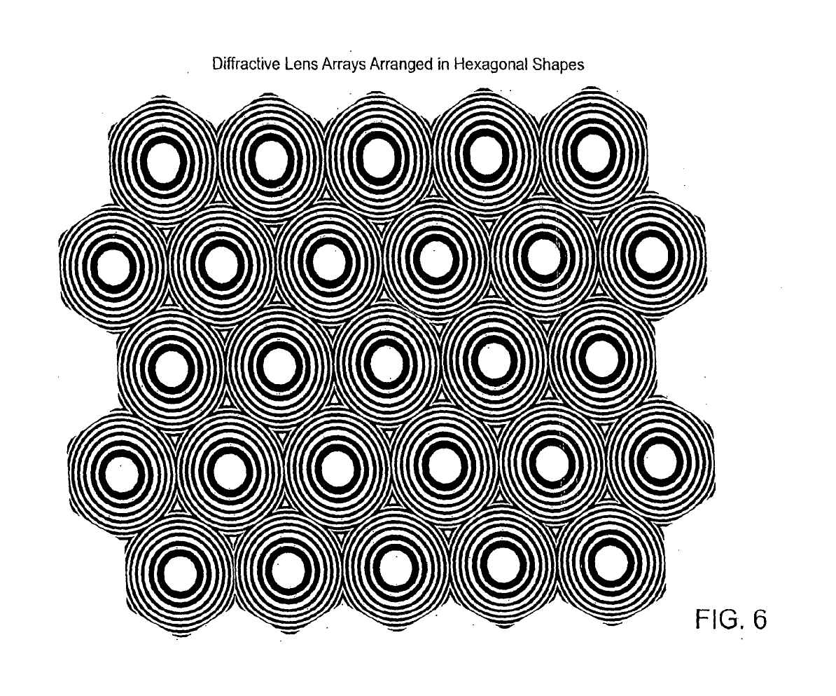

Turning now to FIGS. 3-8, yet another embodiment of the invention is illustrated which uses a diffractive lens array to provide non-uniform heating in the target tissue. A multilevel diffractive lens consists of a number of concentric rings made of optically transparent material with variable thicknesses. The top surface of each concentric ring is flat so the refractive effects are negligible. The variable-thickness rings give rise to a spatial-phase delay pattern on a propagating incident optical beam. The propagating optical beam carries the spatial phase delay pattern past the plane of the diffractive lens and produces an illumination pattern of spatially variable optical intensity. The optical intensity is high at geometrical points that meet the conditions for constructive interference and low at the points that meet the conditions for destructive interference. In general the design of a diffractive lens is optimized so that the principal diffraction maximum (or minimum) would be on the optical axis at a distance from the plane of the lens. The distanced is the focal length of the lens. In general the goal of the diffractive lens design is to increase the fraction of the incident power in the principal diffraction maximum. However, that fraction is always less than 1 depending on the number of levels, the F-number of the lens and other design parameters. In fact, it is possible to design the diffractive lens pattern so that any fraction (less than 1) of the incident power would be in the principal maximum and the rest of the power would be distributed in the secondary maxima.

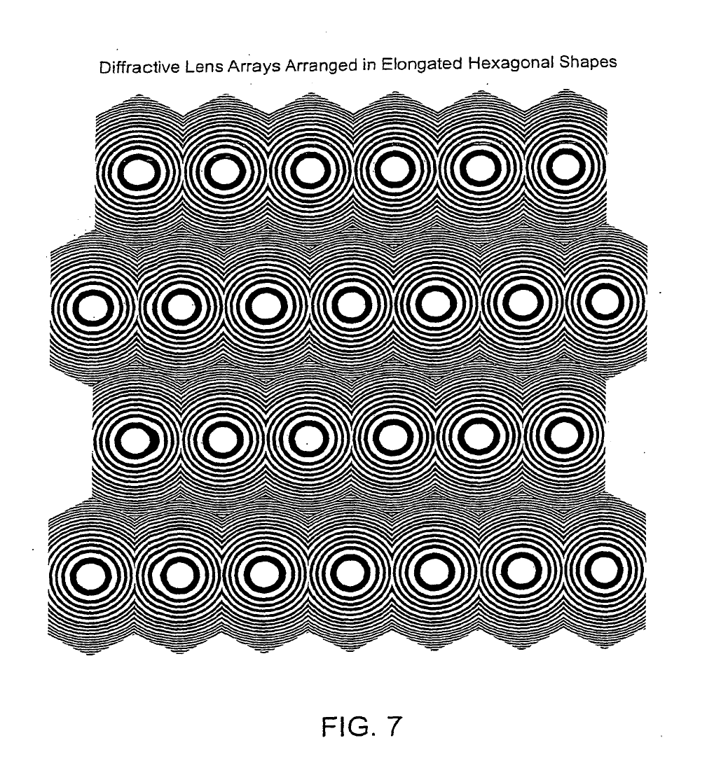

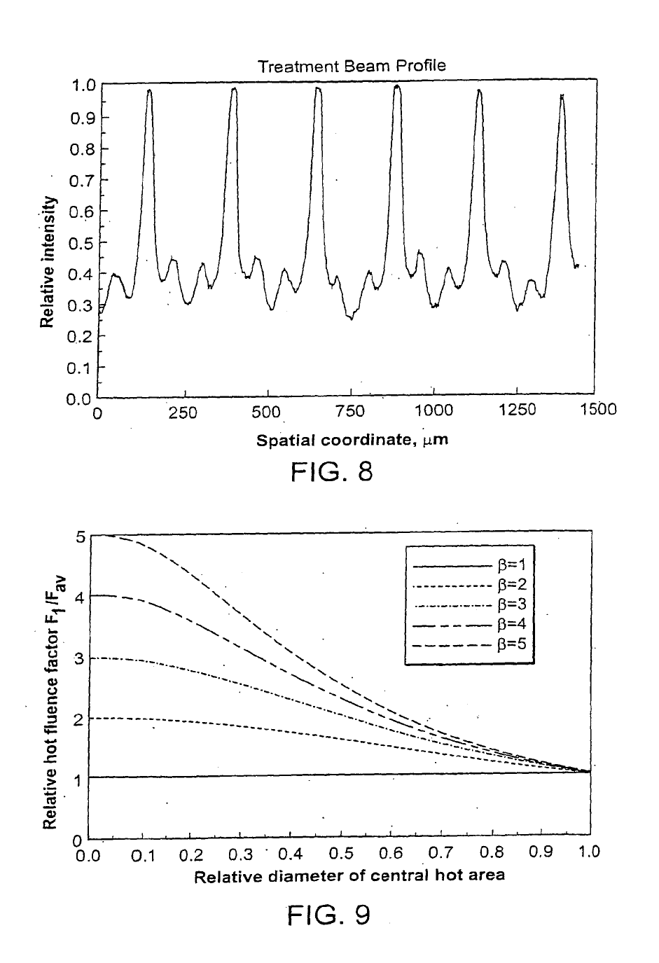

Various examples of multi-level diffractive lenses are shown in cross-sectional views in FIGS. 3-5. FIG. 3 shows a diffractive lens having four levels; FIG. 4 shows a diffractive lens having two levels; and FIG. 5 shows a diffractive lens with eight levels.

In one embodiment of the present invention, a laser treatment apparatus and method utilizes plurality of diffractive lenses that are arranged in an array to produce an output beam having a non-uniform energy profile. More specifically, the diffractive lens array is arranged in an optical path between a laser source and the treatment area, such that each lens in the array provides for an area of higher-fluence “hot spots” surrounded by lower-fluence regions of radiation. In a skin rejuvenation treatment, for example, the higher-energy areas provide sufficient heating to damage and shrink collagen in the “hot spots,” while the lower-intensity radiation regions outside of these hot spots overlap and combine to stimulate collagen regrowth over the entire treatment area.

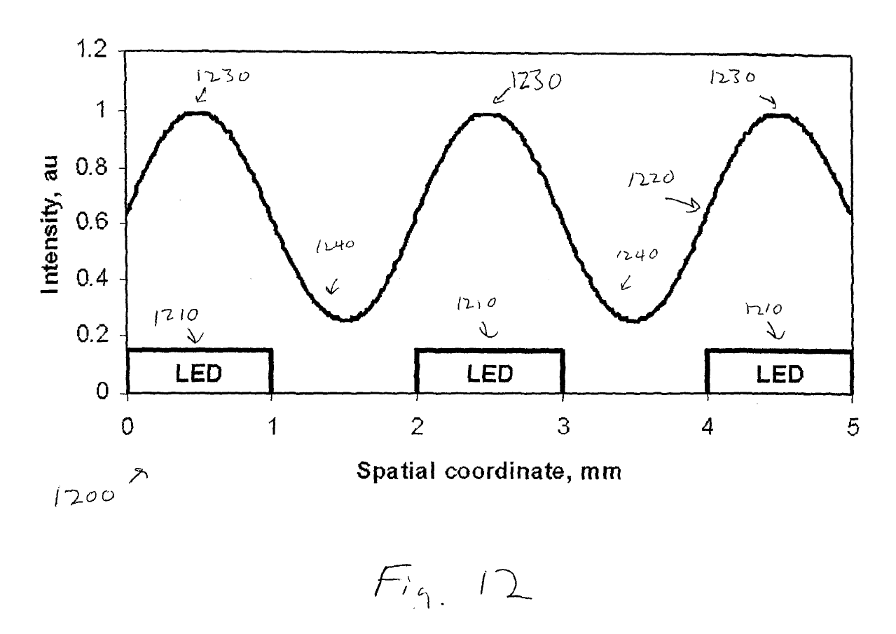

In this embodiment, the laser source preferably produces a pulse of radiation having a wavelength between approximately 1.3 and 1.6 microns, preferably between 1.40 and 1.44 microns, and a-pulse duration of between about 0.1 and 100 milliseconds, preferably between 1 and 5 milliseconds. The laser source can be an Nd:YAG laser, for example. An optical system carries the beam from the laser source to the treatment area. The diffractive lens array is preferably arranged at the distal end of the optical system, adjacent to the patient's skin. The array comprises a plurality of separate diffractive lenses adjacent to one another. In general, there are 2000 or less lenses in an array, and preferably about 1800 lenses. Each lens is between about 150 and 450 microns in diameter, and is preferably about 250 microns in diameter. The entire array of diffractive lenses is generally about 7 to 10 mm in diameter. The array directs the input beam from the laser source (which is preferably also about 7-10 mm in diameter) into a plurality of higher-intensity “hot spots,” corresponding to the central portion of each individual lens in the array, and lower intensity regions surrounding each hot spot. The combined effect in the patient's tissue is to produce a plurality of higher-intensity zones in the skin corresponding to the center of each diffractive lens surrounded by areas of lower-intensity radiation. This is shown in the treatment beam profile of FIG. 8. As can be seen in this graph, the entire treatment area receives at least a low level of treatment radiation, with certain spaced-apart portions receiving a higher dose of laser radiation. In the case of skin rejuvenation, for example, the laser energy penetrates deep into the collagen layer, where the collagen is heated to shrinkage temperatures in the “hot spots,” while the entire treatment area is treated to effect collagen regeneration. In addition to skin rejuvenation treatment, the diffractive lens array can be optimized for use in other applications, such as treatment of acne and hair removal. A different beam profile from the diffractive lens array can be used for different applications.

The diffractive lens is considered to be irradiated by an average uniform fluence, Fₐᵥ, determined by the laser fluence setting selected by the user. In general, the average fluence of the laser in this embodiment is less than about 10 J/cm², and is preferably about 9 J/cm². For purposes of illustration, each diffractive lens with diameter D is assumed to have a simplified design so that it produces a hot area with diameter, d, assumed to have uniform fluence, F₁, and a periphery having a uniform fluence, F₂. The lens design is assumed to produce a fluence ratio, β, of the hot area versus the periphery, β=F₁/F₂. Under these simplifying assumptions, is it possible to derive a simple formula to approximate the hot area fluence, F₁:

$$\frac{{F}_{1}}{{F}_{av}} = \frac{1}{{( \frac{d}{D} )}^{2} + \frac{1}{β} [ 1 - {( \frac{d}{D} )}^{2} ]} .$$

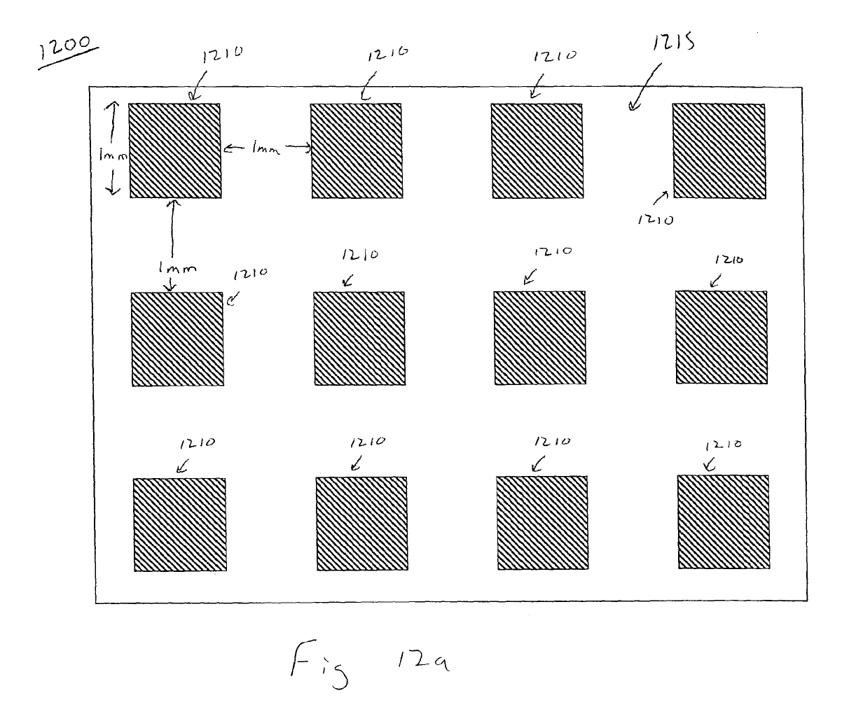

FIG. 9 shows a plot of the relative hot area fluence factor, F₁/Fₐᵥ, as a function of the relative diameter of the central hot area, d/D. As an example, if the diffractive lens is designed to have .beta.=5, with diameter D=250 μm, hot area diameter d=100 μm, and the laser is selected to have average fluence Fₐᵥ=9 J/cm², then the hot area fluence is F₁=3.05×9 J/cm²=27.4 J/cm².

It is to be understood that in various embodiments, the profile of the non-uniform laser beam consists of a plurality of high intensity regions embedded in a low intensity background. Although not seeking to be bound by theory, as described above, the non-uniform distribution is described by high intensity fluence F₁ and a nearly uniform background fluence F₂. In various embodiments, lens designs (e.g. choice of lens performance and relative diameter) may be chosen to produce any desired fluence ratio β of the peak intensity area versus the periphery,

$$β = \frac{{F}_{1}}{{F}_{2}} .$$

The relationship between lens performance ξ, and relative diameter and the fluence ratio β can be described by

$$β = \frac{\frac{1}{{( \frac{d}{D} )}^{2}} - 1}{\frac{1}{ξ} - 1} .$$

In various embodiments lens performance ξ may be e.g. about 40%, about 50%, about 70%, or even about 90% or more, about 95% or more, or about 98% or more. In various embodiments β may be e.g. about 2 or more, about 5 or more, about 10 or more, about 50 or more, about 100 or more, about 150 or more. In some embodiments lenses in the array may have differing) performance values ξ, and the fluence ratio β may vary for different parts of the treatment beam profile.

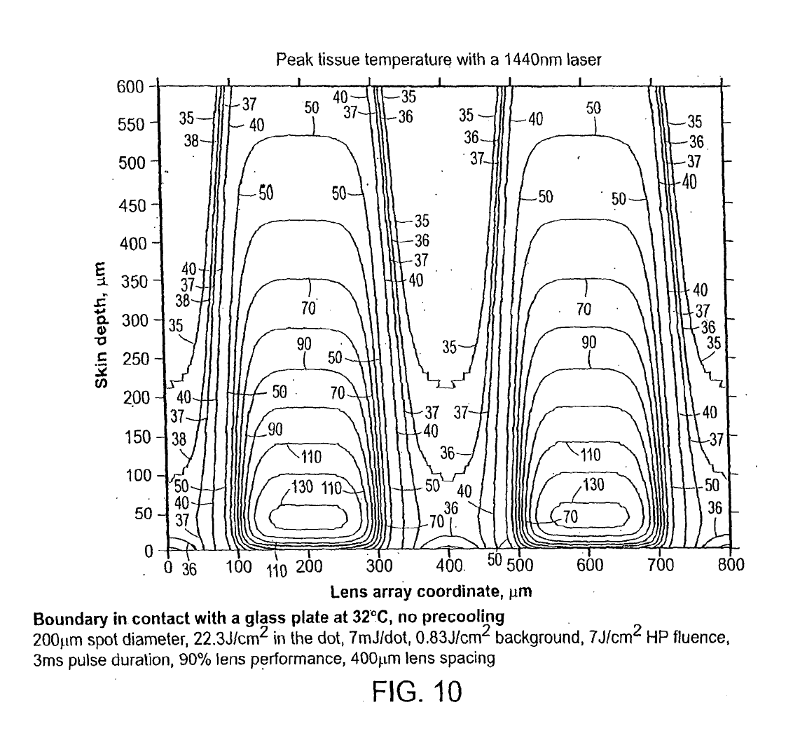

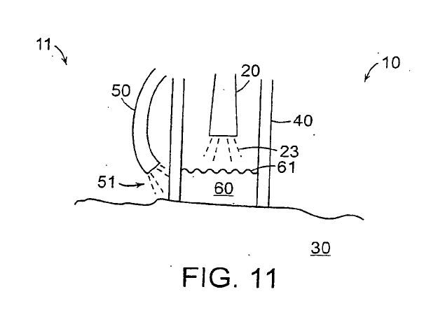

FIGS. 6 and 7 illustrate two exemplary embodiments of a diffractive lens array according to the invention. In FIG. 6, the diffractive lenses are arranged in a hexagonal pattern. In FIG. 7, the lenses are arranged in an elongated hexagonal pattern.

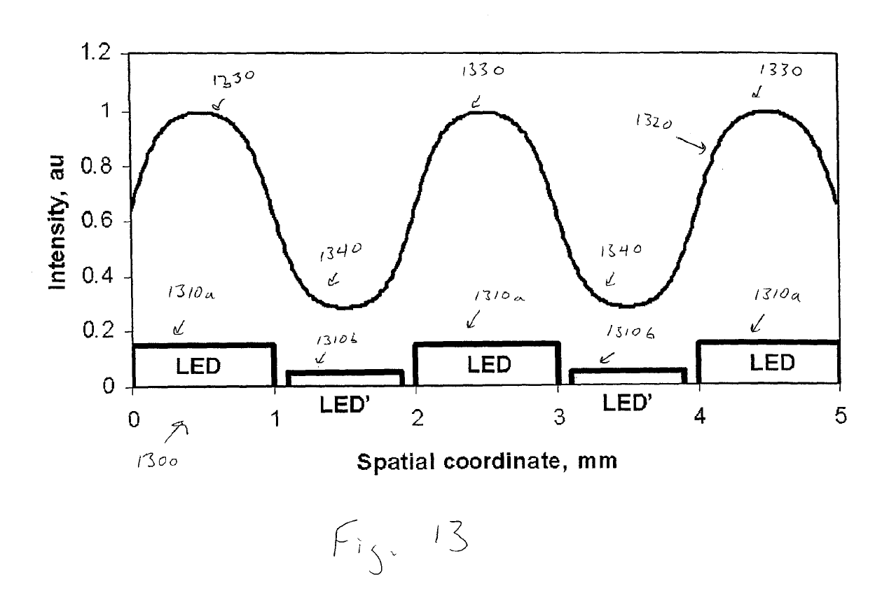

FIG. 10 shows the peak tissue temperature distribution for a portion of skin irradiated with a 1440 nm laser through a diffractive lens array with ξ,=90% lens performance for each lens. As can be seen from the graph, a first diffractive lens is centered at about 200 μm, and a second diffractive lens is centered at about 600 μm on the horizontal axis. As can be seen from this graph, there is an area of tissue about 200 μm wide centered on each of the diffractive lenses that is heated to relatively high peak temperatures (e.g., 70° C. or higher). This high-temperature zone extends from essentially the surface of the skin to a depth of about 350 μm. As discussed above in connection with the fiber-bundle embodiment of FIGS. 1A and 1B, these temperatures are sufficient to cause collagen shrinkage. Outside of these high-temperature treatment zones, the peak temperatures quickly drop off. For example, in the area between about 300 μm and 500 μm on the horizontal axis, the peak skin temperatures are generally between 35° C. (or-less) and 50° C., and are generally less than about 40° C. As previously discussed, these lower intensity zones provide collagen stimulation treatment.

In some embodiments, the temperatures in the treatment zones may be sufficiently high to cause tissue ablation. In some cases, ablation of the high fluence regions leads to a stronger effect of collagen shrinkage compared to the non-ablative collagen destruction and shrinkage achieved in the above described non-ablative mode. For a given application, the fluence needed to achieve ablation is wavelength and pulse duration dependent. Suitable laser sources with high absorption in tissue include the 1440 nm Nd:YAG, the 1940 nm Tm:YALO₃, 2010 nm Tm:YAG, and others.

As an example an Er:YAG laser at 2940 nm will be considered. Not wishing to be bound be theory, in some applications a precise description of the laser ablation process would require a dedicated mathematical model and tissue model. The description of the laser ablation process is complicated because of the dynamic change of the tissue absorption coefficient versus the deposited energy density and the obscuration of the laser beam by the ejected ablation plume. A relatively simple model as described in Vogel et al, Mechanisms of Pulsed laser Ablation of Biological Tissues 103 Chem. Review. 577-644 (2003) shows that for an Er:YAG laser source at 2940 nm the ablation threshold is between 1 and 2.5 J/cm². For laser fluences close to the threshold (between 1.5 and 3 J/cm²) the tissue ablation depth is between 1 and 5 μm.

In some clinical treatment scenarios with the goal of collagen shrinkage, the removal of less than 5 μm of tissue from the high fluence regions may not be sufficient to achieve the desired clinical effect. In such cases, various techniques may be used to increase the ablation depth. One is to increase the fluence, another is to deliver a sequence of pulses with fluence that is slightly higher than the ablation threshold and accumulate the ablation depth. A pulse sequence offers the added benefit that the ablation plume can be taken away, e.g. by a smoke evacuator in the time between the individual pulses in the sequence.

The requirement for fluence between 1.5 and 3 J/cm² in the high fluence regions F₁ can be used to calculate the average fluence Fₐᵥ as described herein. The following table lists parameters for three possible lens array examples. In all cases it is assumed that the fluence in the high fluence region is F₁=3 J/cm² and that the individual diffractive lens in the lens array has 80% performance i.e. 80% of the energy that passes though the lens aperture is delivered in the high fluence region.

| D, μm | d, μm | β | Fₐᵥ, J/cm² | F₂, J/cm² |
|---|---|---|---|---|
| 500 | 200 | 21 | 0.60 | 0.14 |
| 1000 | 500 | 12 | 0.95 | 0.25 |
| 1000 | 350 | 28 | 0.46 | 0.11 |

A clinical treatment with a non-uniform treatment beam that leads to ablation in the high fluence regions would benefit from an optical beam path arrangement where the last optical surface is as far away from the skin surface as possible (e.g. to avoid contamination by the ablation plume). A large skin to optics distance would be beneficial because it decreases the optical contamination from ablated skin fragments. One possible example for such optical arrangement would be a telescope with 2× magnification or more that images the non-uniform beam distribution produced by the diffractive lens array on the skin surface. In other embodiments, any other optical delivery scheme know in the art may be used.

Note that while the examples above for non-uniform treatment beam that leads to ablation in the high fluence regions were given for diffractive lens arrays, similar considerations would apply to fiber bundles as outlined herein.

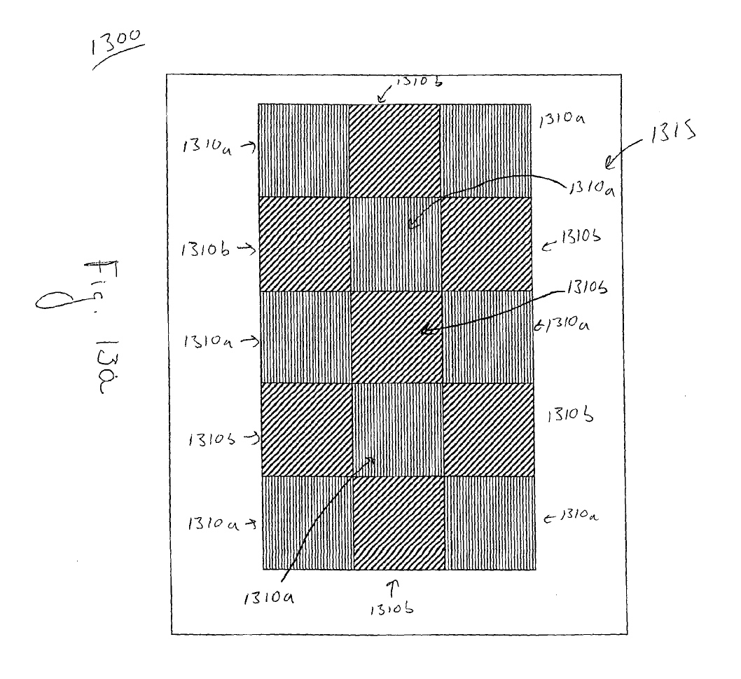

FIG. 11 is a cross-sectional view of a tip 10 of a laser treatment apparatus having a diffractive lens array for providing an output beam having a non-uniform energy profile. The operator applies the tip 10 directly against the patient's skin 30. A laser source (not shown) is energized to produce an output beam 23, and the output beam is carried to the tip 10 by an optical fiber 20. The output beam 23 is emitted from the end of optical fiber 20, and is directed to diffractive lens array 61. Adjacent to the diffractive lens array 61 is an optical window 60 that directly contacts the patient's skin 30. The optical window 60 is similar to the optical window described in connection with FIG. 1, and functions as a spacer between the output end of the fiber bundle and the skin of the patient. The optical window 60 can be integral with the diffractive lens array 61. Preferably, the window is made of a good thermal conductive material, such as glass. The optical fiber 20, lens array 61, and optical window 60 are all enclosed in a tip housing 40, which is preferably a cylindrically-shaped housing. The tip housing 40 can be made of plastic. Outside the tip housing 40 is a cooling mechanism 11. Preferably, the cooling mechanism 11 comprises a conduit 50 that carries cooled air 51 from a cooled air source (not shown) to the tip 10 of the D treatment apparatus. The conduit 50 preferably includes an outlet that is angled with respect to the tip housing 40, so that cooled air 51 is directed at the distal end of the tip housing 40 (i.e. where the tip 10 interfaces with the patient's skin 30). This arrangement provides effective cooling of the skin during laser treatment. Although the tip 10 and cooling mechanism 11 are shown here in connection with the diffractive lens array embodiment of FIGS. 3-8, it will be understood that this design may also be employed with a laser apparatus having a fiber bundle, such as shown and described in connection with FIGS. 1 and 2.

In the embodiments described above, a non-uniform output beam is delivered from a laser source and used for treatment. In some embodiments, it is possible to deliver a non-uniform output beam from non-laser (i.e. at least partially incoherent) sources of electromagnetic radiation (EM) as well. In various embodiments, such sources could include light emitting diodes (LED), pulsed lamps, micro-ring resonators or other emitters of electromagnetic radiation. Using techniques known in the art, the output of any one of these sources can be engineered to consist of a plurality of high intensity zones surrounded by a relatively lower intensity nearly uniform background.

Any of the techniques described above for producing non-uniform output beam may be similarly applied to non-laser EM sources. For example, a fiber bundle or diffractive lens array may be used with a non-laser source as long as the diameters of the fibers in the fiber bundle or the diffractive lenses in the diffractive lens array are relatively small so that they select only a small portion of the output beam of the source.

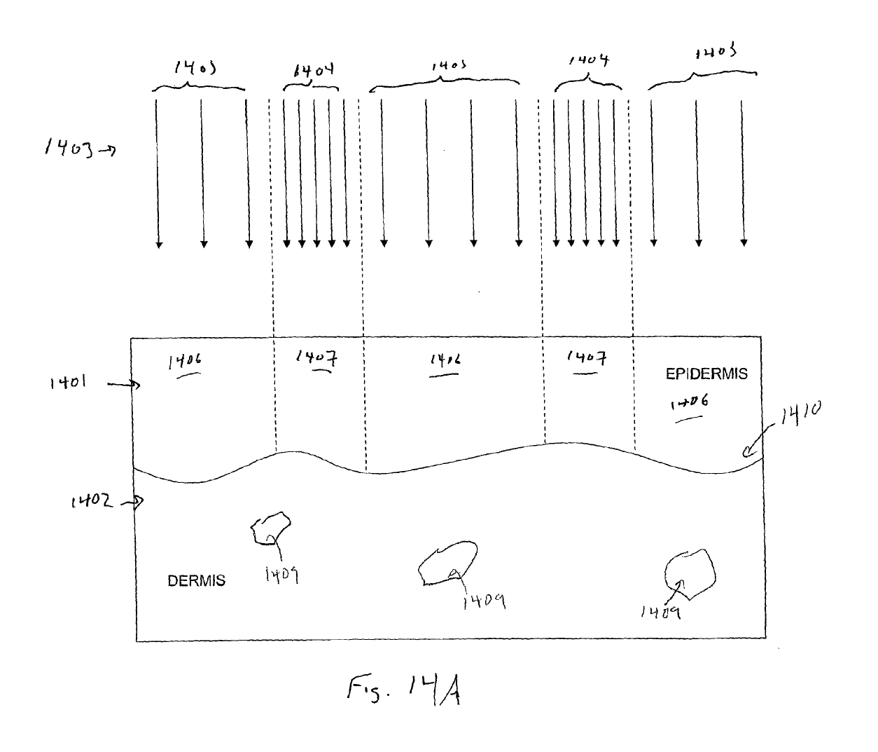

Referring to FIGS. 12 and 12a, an array 1200 of LED chips 1210 can be mounted on a heat dissipating substrate 1215 using, for example, the standard “chip on board” technology known in the art. Also shown in FIG. 12 is a plot of the intensity distribution 1220 of the output beam from array 1200 at a treatment plane. The output beam includes high intensity zones 1230 surrounded by low intensity zones 1240. The size and position of each LED chip 1210 determines the size and position of the high intensity zones 1240. Light emitted in a wide solid angle by each LED will produce the surrounding low intensity zones 1240. As shown, the LED chips 1210 are positioned in array 1200 at such distance from each other so that there is partial overlap of the low intensity zones 1240. In one embodiment, the array 1200 consists of square LED chips 1210 with 1 mm long sides positioned on a rectangular grid with 2 mm increment. The plotted intensity distribution 1220 was calculated in a treatment plane at a distance 0.5 mm from the LED array. In some embodiments, the use of high performance LED material allows the use of smaller chips positioned on a tighter grid. In various embodiments, characteristics such as grid spacing, LED chip size, LED output intensity, etc. are determined by the treatment application at hand and the physiological response of the treated tissue.

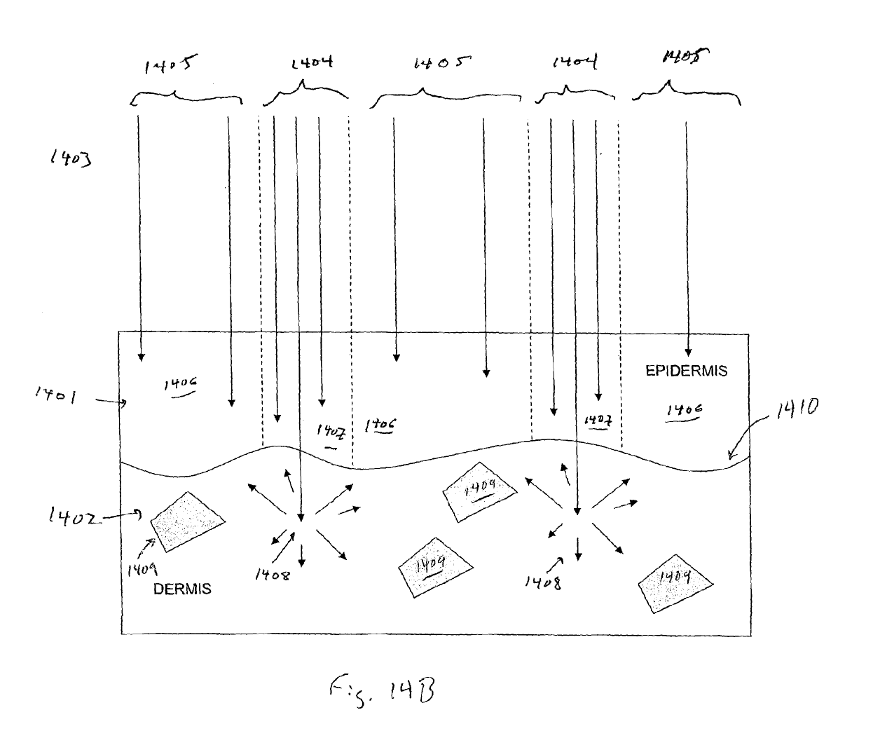

Referring to FIGS. 13 and 13a, a dense packed array 1300 of LED emitter chips 1310a, 1310b is mounted on heat dissipating substrate 1315 using, for example, “chip on board” technology known in the art. The array is dense packed in the sense that LED emitters 1310a, 1310b are separated by gaps which are much smaller than the size of the emitters. High intensity LED emitters 1310a are interspersed with low intensity LED emitters 1310b. Also shown in FIG. 13 is a plot of the intensity distribution 1320 of the output beam from array 1300 at a treatment plane. The output beam includes high intensity zones 1330 surrounded by low intensity zones 1340. As shown, the plotted intensity distribution was calculated in a treatment plane at a distance 0.25 mm from the LED array. In various embodiments, characteristics such as the number relative placement of high and low intensity LED emitters 1310a, 1310b, LED chip size, LED output intensity, etc. are determined by the treatment application at hand and the physiological response of the treated tissue. In typical embodiments, the resulting intensity distribution from array 1300 may be more flexible controlled, but may be more costly because of the dense LED mounting and associated more difficult cooling.

Note that although two exemplary LED arrays are presented above, other suitable arrays may be used with any number of LED emitters arranged in any suitable pattern. In various embodiments, LED emitters of any suitable sizes or shapes may be used.

In typical applications, the non-laser EM sources for non-uniform treatment beam may have a lower brightness than a laser source and therefore may require various modifications in the corresponding patient treatment protocols. For example, a non-uniform treatment with a laser source may produce a cosmetically significant treatment outcome in 1 to 5 treatment sessions and after each treatment it is possible to observe histologically significant tissue effects in the treatment area (collagen modification, tissue inflammation, fibrosis, etc.). Alternatively, a non-laser EM source may require multiple (e.g. greater than 5) treatments with a repeated accumulation of the tissue response.

Some embodiments may employ, a two (or more) step treatment schedule where the first few treatments are done with a laser source to produce the desired tissue response and then a (typically longer) series of maintenance treatments are performed with a non-laser EM source with the said non-uniform beam.

In some embodiments, a treatment system employing a non-laser EM source with a non-uniform beam described may be more appropriate for home use than a laser due to, for example, size, cost, ease of use, maintenance, and/or safety considerations. For example, an LED device may be manufactured to be small, portable, safe, and easy to use with a lower purchase and operating cost for the consumer.

In the embodiments described above, a non-uniform output beam is delivered from a laser source and used for treatment. In some embodiments, it is possible to deliver a non-uniform output beam from non-laser (i.e. at least partially incoherent) sources of electromagnetic radiation (EM) as well. In various embodiments, such sources could include light emitting diodes (LED), pulsed lamps, micro-ring resonators or other emitters of electromagnetic radiation. Using techniques known in the art, the output of any one of these sources can be engineered to consist of a plurality of high intensity zones surrounded by a relatively lower intensity nearly uniform background.

For some applications, it may be desirable to deliver treatment light to a layer of tissue that is covered by another layer, preferably while minimizing or eliminating any deleterious side effects in the covering layer. For example, in some cases, delivery of therapeutically effective doses of treatment light through the covering layer to the underlying layer may result in overheating and damage to the overlaying layer due to absorption of a portion of the treatment light.

For example, a number of dermatological conditions require energy delivery at various depths in the dermis. In most cases it is desirable to preserve the covering epidermis. In some applications, epidermal protection is provided through epidermal cooling e.g., by blowing cold air, or using a cryogen or contact cooling. In some cases where the treatment light is a pulsed laser beam, the laser pulse duration may be adjusted to so that the epidermis cools much faster than the targeted dermal structure. In general, one technique protect the epidermis is to decrease the amount of energy delivered through the epidermis. However a reduced energy delivery may lead to decreased efficacy of treatment. The inventors have realized that this disadvantageous trade off can be avoided using a spatial intensity modulated beam of the type described herein to deliver energy to the tissue.

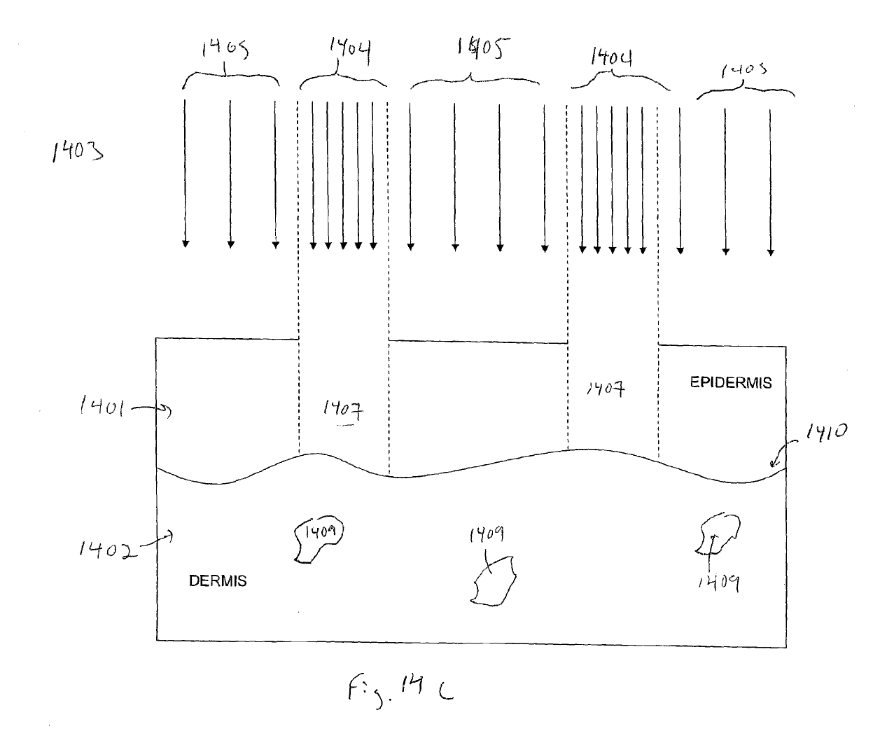

Referring to FIG. 14A, an upper layer of tissue 1401 (as shown, the epidermis) overlays a lower layer of tissue 1402 (as shown, the dermis). A beam 1403 having a non-uniform profile (e.g., generated using any of the techniques and devices described herein) is directed to the surface of the upper layer 1401. The profile of beam 1403 includes areas of relatively high intensity 1404 surrounded by a background of relatively low intensity 1405. The relatively low intensity light impinges on the epidermis and is partially or completely absorbed by the upper layer before reaching the lower layer and without causing any substantial damage to the corresponding portions 1406 of the upper layer. The relatively high intensity light impinges on the epidermis and at least a first portion is transmitted through the upper layer to reach the lower layer. A second portion of the high intensity light is absorbed by the portions of upper layer, which is damaged (e.g. by ablation, denaturing, or any other thermal or optical effect). Because the high intensity light is localized in the beam profile, the damaged portions of the upper layer 1401 will be formed as sacrificial channels 1407 of damaged tissue extending at least partially through the upper layer. For example, in the case where the high intensity regions 1404 of the beam 1403 or circular in shape (thereby impinging on the upper surface 1407 in spots) the sacrificial channels 1407 will be shaped as vertically oriented cylinders extending through the upper layer 1401.

Accordingly, damage in the upper layer 1401 is localized to the sacrificial channel 1407. In some embodiments, where the volume and surface fraction of the damaged zones is kept sufficiently low (e.g. less than 20% of the surface, less than 10% or the surface, less than 5% of the surface, etc.), the upper layer may experience few or no significant side effects. Moreover, in some embodiments, the low intensity background light 1405 delivered to portion 1406 of the upper layer may stimulate healing in the layer which quickly repairs the damage found in the sacrificial channels 1407. For example, in cases where the upper layer is the epidermis, the low intensity light may heat the tissue to stimulate collagen production thereby enhancing the healing of the sacrificial channels.

Referring to FIG. 14B, the fraction 1408 of the high intensity light that passes through the sacrificial channels 1407 in the upper layer 1401 without being absorbed is scattered as it propagates through the lower layer 1402. Propagation in depths larger than a few scattering lengths will lead to overlapping of the scattered photons passing through the individual sacrificial zones. Accordingly, if the penetrating fraction 1408 propagates a sufficient distance through lower layer 14B, it can be scattered to provide nearly uniform delivery of energy through regions deep within the layer (e.g. the deep dermis portion of the dermis).

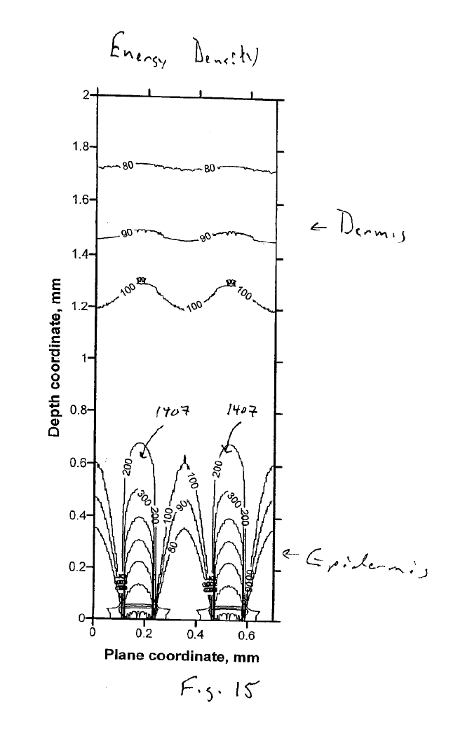

For example, FIG. 15 shows an energy density plot for the delivery of a spatially modulated 1320 nm laser pulse to the surface of the skin. Contours show the delivered energy density as a percentage of a desired therapeutic density. The high energy density sacrificial channels 1407 extend to around 0.6 mm. The scattering in the dermis leads to nearly uniform energy deposition at depths larger than 1.2 mm. In the volume between the sacrificial zones at depths less than 0.6 mm the deposited energy density is less than the therapeutic 100% level (e.g. less than 80%). The lower energy density in these regions makes spares the volume between the sacrificial plugs from damage while treating the deeper dermal layers.

In the case, e.g., of a 1320 nm laser, the clinical application might be bulk deep dermal heating. In that case there are no specific discrete targeted structures in the dermis.

In some embodiments, the delivery of a spatial intensity modulated beam would also benefit the treatment of conditions associated with discrete targets 1509 in the lower layer 1402 (e.g. dermal targets). The discrete targets may include, for example a foreign body (e.g. a tattoo ink particle), a sebaceous gland, a hair follicle, a blood vessel, a region of lipid rich tissue, etc. It is desirable to deliver treatment light (e.g. with a property such as wavelength or pulse duration) chosen so that the energy is preferentially absorbed by the discrete targeted structures 1409, e.g. to heat them more effectively. However, the choice of, e.g., wavelength and pulse duration optimized for preferential treatment of the targeted structures may required radiant exposures leads to various side effects due to absorption by the upper layer (epidermal absorption.) For example in the case of laser tattoo removal, the very short (tens of nanoseconds and shorter) laser pulses could lead to epidermal damage that sometimes is associated with pinpoint bleeding and/or melanosomal damage leading to hyper- or hypo-pigmentation.

As described above, in such cases treatment light optimized to the target structures may be delivered using a non-uniform beam 1403 to the lower layer 1402 (e.g. the dermis) through sacrificial channels 1407, thereby reducing or eliminating unwanted side effects. In a typical treatment the depth of the targeted structures 1409 is well known, but their exact positions are random. In such cases it may be beneficial to deliver nearly uniform fluence density at the targeted depth in the lower layer to allow the randomly positioned discrete targets to absorb the energy preferentially. As noted above, the fraction 1408 of treatment light transmitted through the sacrificial channels to the lower layer is scattered to provide substantially uniform illumination in a region of the lower layer 1408. Accordingly, substantially uniform illumination of the target structures 1409 (e.g. tattoo ink particles) may be achieved.

Referring to FIG. 14C, in some embodiments, the high intensity regions 1404 of the non-uniform beam 1403 may provide sufficient heating to ablate the material in sacrificial channels 1407. The ablated channels 1407 can extend down to a desired depth in the upper or lower layers 1401 and 1402. For example, as shown, the ablated channels 1407 extend down to the interface 1410 of the upper and lower layers, thereby exposing the top surface of the lower layer 1402. In other embodiments, the open ablated channels 1407 may extend down to a desired depth into the lower layer 1402. As in the examples above, in some embodiments, the low intensity background light 1405 heats the tissue 1406 surrounding the ablated channels to stimulate wound healing and collagen re-growth.

In some embodiments, the ablation of the channels 1407 may be accomplished with light at a first wavelength, and treatment light at a second wavelength can be subsequently applied through the open channels 1407 to the lower layer 1402. In some embodiments, the first wavelength may be more preferentially absorbed by the first layer 1401 than the second wavelength. For example, the ablation can be done using a first wavelength with high absorption in tissue—for example around 3 μm, 1.95 μm or between 6 and 12 μm. The treatment can be done with a second wavelength with a lower absorption rate in tissue, e.g., in the range between 300 and 1800 nm. The second treatment wavelength can be chosen based on the absorption characteristic of the targeted structures 1409. The depth of ablation of the sacrificial channels may be determined based on the scattering coefficient of the second wavelength and the depth of the targeted structure 1409 in tissue—e.g. tattoo particles, or hair follicles, or deeper dermal layer, a fat layer etc.

In some embodiments the scattering and absorption of energy from the second wavelength by tissue 1406 between the sacrificial conduits 1407 creates a background heating that stimulates collagen production and speeds up the healing of the sacrificial conduits. In principle, the second wavelength may be followed by a third etc with each wavelength targeting a specific depth and dermatological condition or delivering energy to the tissue 1406 in the space between the sacrificial channels 1407 and speeding healing times and/or improving skin appearance.

In some embodiments, the light at the second wavelength may be applied uniformly across the surface of upper layer 1401. In other embodiments, the light at the second wavelength may be applied using a non-uniform beam, e.g. with high intensity regions aligned with the sacrificial channels.

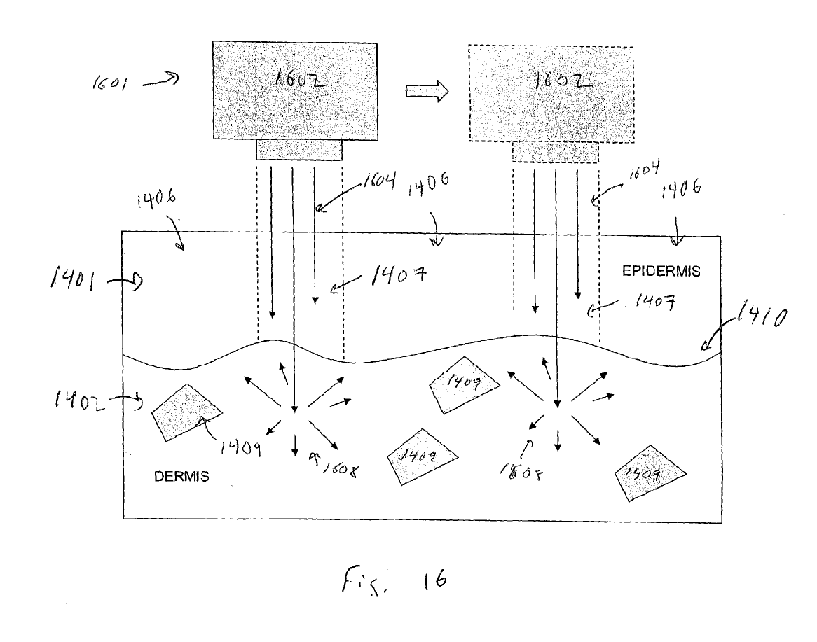

Referring to FIG. 16, treatment schemes of the type described above may be carried out using an optical scanner 1601 to sequentially apply light to localized regions on the surface of the skin, thereby replacing the simultaneously applied high intensity zones of the non-uniform beam 1403 (e.g. generated by a diffractive lens array. For example, as shown, scanner 1601 has an optical delivery head 1602 which can be selectively located at different points above the upper layer 1401. The head directs a light beam to through the first surface forming a sacrificial channel 1407. In some embodiments, a fraction 1608 of the beam 1604 is transmitted through the channel 1407 to the lower layer 1402. As described in detail above, this fraction may be scattered in the lower layer 1402 to provide substantially uniform illumination of target structures 1409.

Optical delivery head 1602 may then be repositioned (e.g. manually, or automatically using a controller) and the process repeated. In some embodiments, the head may be rapidly cycled) through a set of positions, providing a substantially similar illumination pattern to that of a non-uniform beam. Light can be provided to the scanner 1601 from one or more sources using, e.g., an optical fiber or one or more optical elements.

In other embodiments, movable delivery head 1602 of optical scanner 1601 may be replaced by one or more stationary optical elements which can selectively direct light to a sequence of locations on upper layer 1401. The optical elements may include, for example, an articulated lens or mirror, a MEMS device, a digital light processor, an acousto-optic modulator, a rotating lens or mirror, a deformable lens or mirror, a diffractive element, or any other suitable scanning element or elements know in the art.

In some embodiments, optical scanner 1601 is used to deliver sequentially two light beams at a respective first and second wavelength. The first wavelength is chosen to be with very high absorption in tissue (for example from a laser such as an Er:YAG or CO₂ laser). The pulse duration and energy delivered in each sacrificial channel region 1407 by the first wavelength is set to be sufficient to achieve localized tissue ablation extending, e.g., to or beyond the dermal/epidermal interface 1410 to create an open ablated sacrificial channel which admits energy into the lower layer 1402. The second wavelength is chosen to have moderate to high absorption in tissue and relatively high scattering (for example 1320 nm Nd:YAG laser light). The energy delivered by the second wavelength through each sacrificial channel 1407 is set so that, based on tissue scattering and absorption, the energy density at the targeted tissue depth (e.g. 0.2 to 2 mm) is nearly uniform and similar to the energy density that would be delivered in a uniform beam at a level required for therapeutic response. The second wavelength may be followed by a third etc with each wavelength targeting a specific depth and dermatological condition or delivering energy in the space between the sacrificial conduits and speeding healing times and/or improving skin appearance.

In some embodiments, treatment can be done using a first wavelength with high absorption in tissue—for example around 3 μm, 1.95 μm or between 6 and 12 μm. The second wavelength may be in the range between 300 and 1800 nm and can be chosen based on the absorption characteristic of the targeted structures 1409 in the lower layer 1402 and its capability to provide background heating between the ablated conduits and stimulate collagen production. The depth of ablation of the sacrificial channels is determined based on the scattering coefficient of the second wavelength and the depth of the targeted structure in tissue, e.g. sebaceous glands, or hair follicles, or deeper dermal layer, or fat layer etc.

As an example the dual wavelength technique may be applied for treatment of acne by targeting the sebaceous glands in the dermis. The sebaceous glands are located at depths between 0.2 and 1 mm. The first wavelength laser source with high tissue absorption is either an Er:YAG or a CO₂ laser. The laser pulse duration (or dwell time) and energy delivered in each high intensity region is chosen so that a sacrificial channel is ablated (0.1 to 1 ms pulse duration 2 to 5 mJ for Er:YAG, 5 to 25 mJ for CO₂). The resulting open sacrificial channel is cylindrical and has a diameter between 0.1 and 0.5 mm and depth between 0.1 and 0.5 mm or deeper. The scanner 1601 directs the ablative laser wavelength to a designated high intensity region on the skin of the face or the back with high prevalence of acne. After the ablation of the sacrificial channel 1407 the scanner maintains its position and within a few milliseconds the second wavelength source (e.g. a 1320 nm Nd:YAG laser) is delivered through the conduit. The majority of the 1320 nm energy is scattered forward and absorbed in a region extending down to 1 mm depth in the skin. A small fraction of the 1320 nm energy is scattered back and absorbed towards the surface of the skin in the dermis and epidermis and contributes to the background heating and neocolagenogenesis. The sequential delivery of energy from the two wavelength sources is repeated as the optical scanner 1601 sequentially points to the designated sacrificial channel regions 1407 on the surface of the skin. The ablation of each individual sacrificial channel is independent of the rest of them. The delivery of the therapeutic 1320 nm wavelength source through the conduits is cumulative. The cumulative effect of the 1320 nm source deliveries contributes to heating of the dermal layer between 0.2 and 1 mm to a temperature that disrupts the functioning of the sebaceous glands without thermal damage to the dermis. Such temperature exists as evidenced by V. Ross, Optical treatments for acne, Dermatol Ther 18 (2005), pp. 253-266. The above technique circumvents the difficulty of delivering enough energy to reach that temperature while maintaining an intact epidermis.

Another example of a dual wavelength system would combine a first wavelength with very high absorption in tissue generated by a pulsed laser (for example Er:YAG or CO₂) and a second wavelength generated by a continuous wave (CW) or quasi-CW laser (for example a diode laser) e.g. in the visible or near IR region. After the delivery of the ablative pulse and formation of the sacrificial channel 1407, the scanner would be stationary for, e.g., a few milliseconds, between 1 and 100 ms, etc. to allow the energy from the CW source to be delivered through the sacrificial channel. In addition, the CW source may be kept on at a reduced power level during the scanner movement to produce additional background heating of the tissue 1406 between the ablated sacrificial channels 1407.

While this invention has been particularly shown and described with references to preferred embodiments thereof, it will be understood by those skilled in the art that various changes in form and details may be made therein without departing from the scope of the invention encompassed by the appended claims.

1.-35. (canceled)

36. A method of treating human tissue comprising a first layer of tissue overlaying a second layer of tissue, the method comprising:
generating a treatment beam having a non-uniform energy profile from a light source, said non-uniform energy profile being comprised of regions of relatively high energy per unit area within background region of relatively low energy per unit area; and
directing the treatment beam to impinge on the first layer of tissue to form one or more sacrificial channels of damaged tissue in the first layer at positions corresponding to regions of relatively high energy per unit area, wherein the sacrificial channels are surrounded by regions of substantially undamaged tissue in the first layer at positions corresponding to regions of relatively high energy per unit area; and
transmitting treatment beam light through the sacrificial channels to the second layer.

37. The method of claim 36, further comprising scattering at least a portion of the treatment beam light transmitted to the second layer to direct the portion of light to locations in the second layer underlying the regions of undamaged tissue in the first layer.

38. The method of claim 36, wherein the second layer of tissue comprises one or more target structures, and further comprising directing at least a portion of the treatment beam light transmitted to the second layer to the target structures.

39. The method of claim 38, wherein the target structure comprises at least one from the list consisting of: a foreign body, a tattoo ink particle, a sebaceous gland, a hair follicle, a blood vessel, and region of lipid rich tissue.

40.-44. (canceled)

45. The method of claim 36, wherein the regions of relatively high energy per unit area comprise about 20% or less of the total area of a cross section of the treatment beam at the first layer.

46. (canceled)

47. A method of treating human tissue comprising a first layer of tissue overlaying a second layer of tissue, the method comprising: generating a first light beam at a first wavelength, the first light beam having a nonuniform energy profile, said non-uniform energy profile being comprised of regions of relatively high energy per unit area within background region of relatively low energy per unit area; and directing the first treatment beam to impinge on the first layer of tissue to ablate tissue in the first layer at positions corresponding to regions of relatively high energy per unit area to form channels extending at least partially through the first layer; generating a second light beam at a second wavelength; directing the second light beam to impinge on the first layer such that a portion of the second light beam is transmitted the channels to the second layer.

48. The method of claim 47, wherein light at the first wavelength is more preferentially absorbed by the first layer of tissue than light at the second wavelength.

49. The method of claim 47, wherein the second light beam has a non-uniform energy profile, said non-uniform energy profile being comprised of regions of relatively high energy per unit area within background region of relatively low energy per unit area, and the step of directing the second light beam to impinge on the first layer comprises directing the second light beam to the first layer such that the regions of relatively high energy per unit area of the second light beam impinge upon the first layer at locations which substantially correspond to the channels in the first layer.

50. The method of claim 47, further comprising controlling the ablation of the tissue in the first layer such that the channels extend substantially through the first layer to a location proximal an interface between the first and second layer.

51. The method of claim 50, wherein controlling the ablation comprises controlling at least one of: an intensity of the first light beam, a pulse period of the first light beam, a pulse rate of the first light beam, a pulse shape of the first light beam.

52. The method of claim 47, wherein the channels are surrounded by regions of substantially undamaged tissue.

53. The method of claim 47, further comprising scattering at least a portion of light from the second beam transmitted to the second layer to direct the portion of light to locations in the second layer which do not underlay the channels.

54. The method of claim 47, wherein the second layer of tissue comprises one or more target structures, and further comprising directing at least a portion of the light from the second beam to the target structures.

55. The method of claim 54, wherein the target structure comprises at least one from the list consisting of: a foreign body, a tattoo ink particle, a sebaceous gland, a hair follicle, a blood vessel, and region of lipid rich tissue.

56. The method of claim 47, wherein the first layer comprises an epidermis of a region of skin and the second layer comprise a dermis of a layer of skin.

57.-60. (canceled)

61. The method of claim 47, wherein the regions of relatively high energy per unit area comprise about 20% or less of the total area of a cross section of the treatment beam at the first layer.

62. An apparatus comprising: an optical scanner configured to selectively direct light to each of plurality of locations of a treatment region comprising a first layer of tissue overlaying a second layer of tissue; a controller configured to, for each of the plurality of locations: direct a first beam of light at a first wavelength from the scanner to the first layer of tissue to ablate a respective channel at least partially through the first layer of tissue; and direct a second beam of light at a second wavelength from the scanner to the first layer of tissue and through the channel to the second layer of tissue.

63. The apparatus of claim 62, wherein light at the first wavelength is more preferentially absorbed by the first layer of tissue than light at the second wavelength.

64. The apparatus of claim 62, wherein the controller adjusts the first light beam such that each respective channel extends substantially through the first layer to a location proximal an interface between the first and second layer.

65. The apparatus of claim 62, wherein the controller adjusts the first light beam such that the respective channels extends through the first layer and to a desired depth in the second layer.

66. The apparatus of claim 62, wherein the controller is adapted to adjust at least one property of the first light beam chosen from the list consisting of an intensity of the first light beam, a pulse period of the first light beam, a pulse rate of the first light beam, a pulse shape of the first light beam, and a wavelength of the first light beam.

67. The apparatus of claim 62, wherein the scanner comprises one or more optical elements which limits the spot size of the first beam such that the respective channels are surrounded by regions of substantially undamaged tissue.

68. A method of treating human tissue comprising a first layer of tissue overlaying a second layer of tissue, the method comprising:
using an optical scanner to direct light to each of a plurality of locations on the first layer of tissue; and
at each location respectively,
directing a first beam of light at a first wavelength to the location to ablate a respective channel at least partially through the first layer of tissue; and
directing a second beam of light at a second wavelength to the location to transmit a portion of the light at the second wavelength through the channel to the second layer of tissue.

69. The method of claim 68, wherein light at the first wavelength is more preferentially absorbed by the first layer of tissue than light at the second wavelength.

70. The method of claim 68, comprising controlling the first light beam such the ablation of the tissue in the first layer such that the respective channels extends substantially through the first layer to a location proximal an interface between the first and second layer.

71. The method of claim 67, comprising controlling the first light beam such the ablation of the tissue in the first layer such that the respective channels extends through the first layer and to a desired depth in the second layer.

72. The method of claim 70, wherein controlling the first light beam comprises adjusting at least one property of the first light beam chosen from the list consisting of an intensity of the first light beam, a pulse period of the first light beam, a pulse rate of the first light beam, a pulse shape of the first light beam, and a wavelength of the first light beam.

73. The method of claim 67, comprising limiting the spot size of the first beam such that the respective channels are surrounded by regions of substantially undamaged tissue.

74. The method of claim 67, comprising forming an array of the channels over an array of tissue, wherein the area of the channels is less than about 20% of the area of tissue.
